(19) United States
(12) Patent Application Publication
(10) Pub. No.: US 2003/0144237 A1
(43) Pub. Date: Jul. 31, 2003

(54) Use of inhibition of a gas6 function or of a gas6 receptor for preventing and treating a cardiovascular disease

(76) Inventors: Peter Carmeliet, Oud-Heverlee (BE); Desire Collen, London (GB); Anne Angelillo-Scherrer, Etoy (CH)

Correspondence Address: William M Lee Jr, PO Box 2786, Chicago, IL, 60610, US

(21) Appl. No.: 10/257,173

(22) PCT Filed: Apr. 13, 2001
(86) PCT No.: PCT/EP01/04312

(30) Foreign Application Priority Data
Apr. 13, 2000 (GB) 0009321.1
Oct. 20, 2000 (EP) 00203668.9

(51) Int. Cl. A61K 48/00; A61K 39/395

(52) U.S. Cl. 514/44; 424/146.1

(57) ABSTRACT

An antagonist to the activator of the Rse and Mer receptor protein tyrosine kinases, encoded by growth arrest-specific gene 6 (gas6), is found to be useful in a method of treating an insulin-resistant disorder such as diabetes. More particularly, a method for treating an insulin-resistant disorder is provided which comprises administering to a mammal in need of such treatment an effective amount of a composition comprising a gas6 antagonist. A hypoglycemic agent may be co-administered with the gas6 antagonist.

The present invention relates to a new method for the prevention and treatment of a thromboembolic disease such as arterial or venous thrombosis based on the inhibition of, e.g. based on the administration of an inhibitor of, a growth arrest-specific gene 6 (Gas6) function or of a Gas6 receptor.

### BACKGROUND OF THE INVENTION

The major factors involved in the patho-physiology of thrombosis are abnormalities of the vessel wall, alterations of blood flow, and changes in the composition of the blood. Arterial and venous thrombosis and their complications, which include focal ischemic cerebral infarction (ischemic stroke), acute myocardial infarction and venous thromboembolism among others, represent the major cause of morbidity and death in the developed countries of the world.

Platelets play a central role in arterial thrombosis. They adhere to exposed subendothelial matrix proteins and become activated. They change their shape and then aggregate. Tissue factor (TF) is thought to be the primary initiator of in vivo blood coagulation. In the absence of TF expression, endothelial cells actively maintain thromboresistance. Vascular wall damage exposes TF which binds activated factor VII (factor VIIa). The factor VIIa-TF complex then triggers thrombin generation by activating factors IX and X. In addition to activating platelets, thrombin converts fibrinogen to fibrin, amplifies its own generation by activating factors V and VIII, and then activates factor XIII which finally stabilizes the fibrin clot, according to Bates et al. in Cardiovasc. Res. (1999) 41:418-432 and Davie E. W. in Thromb. Haemost. (1995) 74:1-6. Prevention and treatment of thrombosis are therefore based on the administration of either antiplatelet drugs or anticoagulants, or of a combination of both.

One of the inherited risk factors for thrombosis is protein S deficiency. Protein S, a vitamin K-dependent plasma protein, serves as a cofactor for the anticoagulant activity of an other vitamin K-dependent protein, activated protein C (APC). The protein C anticoagulant system provides important control of the blood coagulation cascade by degrading coagulation factors Va and VIIIa according to B. Dahlback in Thromb.Haemost. (1991) 66:49-61. Resistance to APC is the most common form of inherited thrombosis disease according to B. Dahlback in Blood (1995) 85:607-614.

In order to investigate the mechanism controlling growth arrest in mammalian cells, a set of six growth arrest-specific (hereinafter “Gas”) genes have been cloned and sequenced. Gas6 was originally identified as a gene whose expression in mouse fibroblasts increased during serum starvation and was described in detail, together with its human homolog, by Manfioletti et al. in Mol. Cell Biol. (1993) 13(8):4976-4985 and U.S. Pat. No. 5,538,861.

The protein encoded by Gas6 is a vitamin K-dependent protein related to protein S (i.e. human Gas6 cDNAs encode a protein having 44% amino acid sequence identity to human protein S) which is suspected to play a role in a number of biological processes, namely the regulation of a protease cascade relevant in cell growth regulation, according to Matsubara et al. in Dev.Biol. (1996) 180:499-510. Both molecules comprise a gamma-carboxyglutamic acid rich region (i.e. the A domain), four epidermal growth factor (EGF)-like repeats (forming the C domain) and a carboxyterminal tandem globular (G) region with homology to the steroid hormone binding globulin (SHBG) protein (i.e. the D domain). However, in contrast to protein S, Gas6 lacks a loop which is crucial for the anticoagulant activity of protein S, according to Manfioletti et al. (cited supra).

The Axl receptor, disclosed by O'Bryan et al. in Mol. Cell Biol. (1991) 11:5016-5031, was identified due to its ability to render mouse fibroblast cells tumorigenic. Axl expression appears to have profound effects on the growth state of cells. U.S. Pat. No. 5,538,861 discloses that Gas6 is a ligand for the Axl receptor. The cDNA sequence of the receptor tyrosine kinase Rse, that is preferentially expressed in the adult brain, was described by Mark et al. in J.Biol. Chem. (1994) 269:10720. cDNA sequences encoding proteins identical to human and human Rse have been termed Sky and Tyro3 respectively and disclosed by Ohashi et al. in Oncogene (1994) 9:699 and by Lai et al. in Oncogene (1994) 9:2567 respectively. Rse is structurally related to Axl (also known as Ufo or Ark) and shares 43% overall amino acid sequence identity with this tyrosine kinase receptor. Rse and Axl, together with c-Mer (also known as Eyk or Nyk) disclosed by Graham et al. in Cell Growth Differ. (1994) 5:647, define a class of tyrosine kinase receptors whose extracellular domains resemble neural cell recognition and adhesion molecules. Like Rse, Axl is also expressed in the nervous system, but is more widely expressed than Rse in peripheral tissues. Gas6 binds members of the above-mentioned class of tyrosine kinase receptors according to Nagata et al. in J. Biol. Chem. (1996) 271(47):30022-30027 and Crosier et al. in Pathology (1997) 29(2):131-135.

The extracellular domains of these receptors comprise two immunoglobulin (Ig)-like repeats followed by two fibronectin type III repeats, found in cell adhesion molecules. The Axl receptor is capable of homophilic binding as well as binding to Gas6. However, Axl is not only expressed as a transmembrane protein, but is also cleaved in the extracellular domain to generate a soluble Axl form, which has been detected in conditioned media of Axl expressing cells, serum, plasma, brain, liver, spleen and tumor cells. Soluble Axl could act as a competitive inhibitor for Gas6 by sequestering free Gas6 or could bind to Axl transmembrane receptor. Binding of soluble Axl to Axl transmembrane receptor might give a signal distinct from Gas6 or inactivate Axl transmembrane receptor on the cell surface according to Costa et al. in J. Cell Physiol. (1996) 168(3):737-744 and Varnum et al. in Nature (1995) 373:623-626.

Gas6 and Axl are expressed by vascular endothelial cells according to Varnum et al. (cited supra). Gas6 has been reported to inhibit homophilic Axl-mediated aggregation of myeloid cells according to Avanzi et al. in Blood (1998) 91(7):2334-2340, but cell-bound Gas6 may mediate aggregation of myeloid cells via interaction with Axl receptor on adjacent cells according to McCloskey et al. in J. Biol. Chem. (1997) 272(37):23285-23291. Gas6 does not affect adhesion of granulocytes to resting endothelial cells, while it inhibits granulocyte adhesion to TNF-α activated endothelial cells at high concentrations according to Avanzi et al. (cited supra). Gas6 is mitogenic for fibroblasts according to Goruppi et al. in Oncogene (1996) 12(3):471-480 and for Schwann cells according to Li et al. in J. Neurosci. (1996) 16(6):2012-9 and U.S. Pat. No. 5,714,385, but not for myeloid cells according to Avanzi et al. in Exp. Hematol. (1997) 25(12):1219-1226 or endothelial cells. Gas6, induced in injured vascular smooth muscle cells, induces Axl-mediated chemotaxis of smooth muscle cells and, although not mitogenic by itself, enhances the mitogenic activity of thrombin according to Fridell et al. in J. Biol. Chem. (1998) 273(12):7123-7126. Gas6 also acts as a survival factor for serum-starved fibroblasts and GnRH neuronal cells, presumably via activation of P13-kinase and Akt kinase according to Goruppi et al. in Mol. Cell Biol. (1997) 17(8):4442-4453. Axl signaling protects against apoptosis as Axl deficient fibroblasts cannot be rescued by Gas6 after serum-withdrawal according to Bellosta et al. in Oncogene (1997) 15(20):2387-2397.

### SUMMARY OF THE INVENTION

In a first aspect, the present invention relates to the use of inhibition of a growth arrest-specific gene (Gas6) function or of a Gas6 receptor (for instance by means of an inhibitor or antagonist such as a Gas6 function neutralizing antibody, or by means of a ribozyme or an antisense RNA directed against Gas 6 or a Gas 6 receptor function) for the manufacture of a medicine for the prevention or treatment of a cardiovascular disease other than resulting from an endothelial dysfunction, e.g. a disease caused by platelet aggregation, in particular a thromboembolic disease or a thrombotic pathologic condition in a mammal, preferably in a human. Within the framework of this invention, the growth arrest-specific gene (Gas6) receptor to be inhibited preferably is a tyrosine kinase receptor such as the Axl receptor, the Rse receptor, the c-Mer receptor or fragments thereof. Inhibition of the Gas6 function may also be effected by means of a protease able to cleave the extracellular domain of the Axl receptor, preferably within the sequence VKEPSTPAFSWPWW.

Inhibition according to this invention also includes inhibition of the native protein or polypeptide encoded by Gas6 or of a modified form thereof, for instance a form including a modified gamma-carboxyglutamic acid rich region (i.e. the A domain)—such as disclosed in U.S. Pat. No. 6,017,882—that enhances membrane binding affinity of the protein relative to the corresponding native protein.

Examples of thromboembolic diseases or thrombotic pathologic conditions within the scope of this invention namely include:

ischemic diseases such as ischemic stroke or ischemic cerebral infarction, acute myocardial infarction, chronic ischemic heart disease.

an ischemic disease of an organ other than myocardium or a region of the brain, for instance a peripheral limb.

venous thromboembolism.

arterial or venous thrombosis.

pulmonary embolism.

restenosis following coronary artery bypass surgery or following percutaneous transluminal angioplasty of coronary artery.

disseminated intra-vascular coagulation.

As far as prevention is concerned, non-limiting examples of thrombotic pathologic conditions within the scope of this invention namely include, in addition to the above:

the relapse of coronary thrombosis after acute myocardial infarction.

coronary thrombosis in patients with unstable angina pectoris.

cerebral ischemic infarction (ischemic stroke) in patients with atrial fibrillation.

arterial thrombosis after vascular surgery.

the occlusion of arterio-venous shunt in dialysis patients.

In a second aspect, the present invention relates to a pharmaceutical composition comprising an inhibitor or antagonist of a Gas6 function or of a Gas6 receptor, or a ribozyme or an antisense RNA directed against Gas 6 or a Gas 6 receptor function, or a protease able to cleave the extracellular domain of the Axl receptor as a first active ingredient in admixture with at least a pharmaceutically acceptable carrier, the said pharmaceutical composition being preferably intended for the prevention or treatment of a cardiovascular disease other than resulting from an endothelial dysfunction, e.g. a disease caused by platelet aggregation, in particular a thromboembolic disease or a thrombotic pathologic condition, such as above defined. The said pharmaceutical composition may further optionally comprise a thrombolytic agent, preferably in respective proportions with the said first active ingredient such as to provide a synergistic effect in the said prevention or treatment.

In another aspect, the present invention relates to the use of inhibition, for instance by means of an inhibitor or antagonist, of a growth arrest-specific gene (Gas6) function or of a Gas6 receptor during extracorporeal blood circulation and hemodialysis, i.e. in a method for treating blood from a mammal, in order to prevent platelet activation leading to thrombus formation in the extracorporeal system and—because of excessive platelet—bleeding in the patient. In yet another aspect, the present invention relates to the use of inhibition, for instance by means of an inhibitor or antagonist, of a growth arrest-specific gene (Gas6) function or of a Gas6 receptor as a diagnostic tool or agent, for instance in order to identify, via protein or mRNA or DNA characterization, individuals having a predisposition to acquire a a thromboembolic disease or a thrombotic pathologic condition, such as above defined.

Finally, the present invention provides a method of prevention or treatment of a cardiovascular disease other than resulting from an endothelial dysfunction, e.g. a disease caused by platelet aggregation, in particular a thromboembolic disease or a thrombotic pathologic condition (such as above defined) in a mammal, preferably a human, comprising administering to a mammal in need of such prevention or treatment a therapeutically effective amount, i.e. preferably an amount able to protect the patient against thromboembolism without causing bleeding side effects, of an inhibitor of a Gas6 function or of a Gas6 receptor, or a ribozyme or an antisense RNA directed against Gas 6 or a Gas 6 receptor function, or a protease able to cleave the extracellular domain of the Axl receptor.

### BRIEF DESCRIPTION OF THE DRAWINGS

FIG. 1 shows the effect of Gas6 deficiency or of anti-Gas6 antibodies on platelet aggregation.

FIG. 2 shows the aggregation of wild-type (+/+) and gas6 deficient (−/−) platelets to thrombin, phorbol-12-myristyl-13-acetate (PMA) and the Ca⁺⁺ ionophore A23187.

### DEFINITIONS

The term “antisense”, as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene of interest in a reverse orientation to a promoter which permits the synthesis of a complementary strand. Once introduced into a cell, this transcribed strand combines with natural sequences produced by the cell to form duplexes. These duplexes then block either further transcription or translation. In this manner, mutant phenotypes may be generated.

“Gas 6 antagonist” refers to a substance that opposes or interferes with a functional activity of Gas6. Examples of Gas 6 antagonists include neutralizing antibodies, Rse-IgG, Rse extracellular domain, Axl-IgG, Axl extracellular domain, Mer-IgG and Mer extracellular domain.

The term “antibody” is used in the broadest sense and specifically covers single monoclonal antibodies against Gas6 or a Gas6 receptor (including agonist and antagonist antibodies) and anti-Gas6 antibody compositions with polyepitopic specificity.

A “monoclonal antibody” is obtained from a population of substantially homogeneous antibodies, except for possible naturally-occurring mutations that may be present in minor amounts. Monoclonal antibodies are highly specific, being directed against a single antigenic site. In contrast to polyclonal antibody preparations, each monoclonal antibody is directed against a single determinant on the antigen. The monoclonal antibodies herein include hybrid and recombinant antibodies produced by splicing a variable (including hypervariable) domain of an anti-Gas6 antibody with a constant domain (e.g. “humanized antibodies) or the like, so long as they exhibit the desired biological activity. “Humanized” forms of non-human antibodies are specific chimeric immunoglobulins or fragments thereof (such as Fv, Fab, Fab′, F(ab′)₂ or other antigen-binding subsequences) which contain minimal sequence derived from non-human immunoglobulin.

“Neutralizing antibody” refers to an antibody capable of substantially (i.e. at least about 50%) inhibiting the functional activity of Gas6, as determined by using an ELISA-based kinase receptor activation assay as disclosed for instance by U.S. Pat. No. 5,955,420.

### DETAILED DESCRIPTION OF THE INVENTION

We have now found that Gas6 deficient mice are resistant to thrombosis, induced by venous stasis, arterial denudation or collagen/epinephrine injection—models known to depend to a variable degree on blood coagulation and platelet aggregation according to Di Minnio et al. in J. Pharmacol. Exp. Ther. (1983) 225:57-60, Herbert et al. in Blood (1992) 80:2281-6 and Matsuno et al. in Br. J. Pharmacol. (1992) 106:533-8. Their resistance to thrombosis was not due to differences in coagulation or thrombolysis, nor to abnormalities in the number or morphology of platelets. Instead, loss of Gas6 caused platelet dysfunction. Indeed, Gas6, while ineffective by itself, significantly enhanced the formation of stable platelet aggregates by several platelet agonists such as adenosine 5′-diphosphate (ADP), collagen and the thromboxane A₂ (TX A₂) analogue U46619. In the absence of Gas6, low concentrations of these agonists could only induce reorganization of actin filaments that cause the shape changes preceding initial formation of platelet aggregates. Signaling by the ADP-, collagen-, TXA₂- or thrombin-receptors was not completely blocked in Gas6 deficient platelets, since shape change did occur in response to low concentrations of the platelet agonists and irreversible platelet aggregation proceeded in response to high concentrations of these agonists. Only thrombin induced aggregation of Gas6 deficient platelets at low concentrations, but adenosine 5′-triphosphate (ATP) release induced by thrombin was lower in Gas6 deficient platelets than in wild type platelets. The downstream pathways mediating granule secretion and platelet aggregation according to Rao et al. in Arterioscl. Thromb. Vasc. Biol. (2000) 20:285-289 were operational in Gas6 deficient platelets, since PMA or the Ca⁺⁺ ionophore A23187 induced normal secretion and aggregation. Thus, in Gas6 deficient platelets both secretion of granules and aggregation of platelets occurred less efficiently.

An autocrine role for Gas6 in platelets is suggested by the finding that Gas6 is present in □-granules and, following platelet activation, becomes secreted and bound to the platelet surface, most likely via Gas6 receptors. Since Gas6 deficient platelets have normal expression of the Gas6 receptors Axl and Sky, the platelet defects were not related to downregulation of these receptors. Collectively, our data are consistent with a model in which Gas6 is released from the α-granules upon initial stimulation of platelets by several agonists. Subsequently, Gas6 amplifies, via signaling through one or more of its receptors, the intracellular signals generated via the ADP-, collagen-, TXA₂- and thrombin-receptors. Gas6 exerts this amplification signal at the level or downstream of the platelet agonist receptors, but likely upstream of protein kinase C activation or Ca⁺⁺ mobilization.

The phenotype of Gas6 deficient mice resembles several features of patients with platelet signal transduction defects. Like Gas6 deficient mice, these patients have impaired secretion of dense granules in response to weak agonists or to low concentrations of potent agonists. The number of platelet granules, TXA₂ production and initial aggregation are normal according to Rao et al. (cited supra). The findings of the present invention therefore suggest Gas6 defects as a mechanism of these primary signal transduction defects.

Gas6 appears to be redundant for baseline hemostasis but constitutes an important “amplification” system in pathological conditions. Because Gas6 only amplifies the response of other platelet agonists, but does not evoke a response itself, inhibition of Gas6 constitutes an attractive treatment to prevent thrombosis without causing bleeding side-effects. Indeed anti-Gas6 antibodies protected wild type mice against fatal thromboembolism to the same degree as genetic inactivation of Gas6, while not causing spontaneous bleeding, implying a therapeutic efficiency of Gas6 inhibitors in the treatment of thrombotic disorders. By modulating—but not completely blocking—signaling of the principal platelet agonists, Gas6 antagonists are believed to be safer than the currently available antiplatelet drugs. Anti-Gas6 antibodies may be obtained by screening test inhibitory compounds from large libraries of synthetic or natural compounds. Synthetic compound libraries are commercially available from e.g. Maybridge Chemical Co. (Trevillet, Cornwall, UK), Comgenex (Princeton, N.J.), Brandon Associates (Merrimack, N.H.), Microsource (New Milford, Conn.) and Aldrich Chemical Company, Inc. (Milwaukee, Wis.). Alternatively, libraries of natural compounds in the form of bacterial, fungal, plant and animal extracts are available from e.g. New Chemical Entities, Pan Laboratories, Bothell, MycoSearch and Chiron Inc. Additionally, synthetic and natural inhibitory compounds obtained from such libraries may be readily modified through conventional chemical, physical and biochemical means. An illustrative example of an anti-Gas6 antibody suitable for the performance of the present invention is constituted by the antibody referenced as 620SC_{—}1935 in the catalogue from Santa Cruz Biotechnology, Santa Cruz, Calif., directed against the carboxy-terminal part of Gas6.

These data show that inhibitors or antagonists of the Gas6 function or of a Gas6 receptor can be used for the manufacture of a medicament for the prevention and/or treatment of a cardiovascular disease caused by platelet aggregation, in particular a thromboembolic disease or a thrombotic pathologic condition, such as arterial and/or venous thrombosis, in a mammal. They appear to constitute a new class of promising antithrombotic drugs with reduced bleeding tendency. In view of a suitable bioavailability, said inhibitors or antagonists are preferably used as active ingredients in pharmaceutical compositions further comprising a pharmaceutically acceptable carrier. Suitable pharmaceutical carriers for this purpose are described for instance in Remington's Pharmaceutical Sciences 16^{th} ed. (1980) and their formulation is well known to those skilled in the art. They include any and all conventional solvents, dispersion media, coatings, antibacterial and antifungal agents (for example phenol, sorbic acid, chlorobutanol), isotonic agents (such as sugars or sodium chloride) and the like. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition comprising the active ingredient may require protective coatings as are well known in the art. The pharmaceutical form suitable for injectionable use include sterile aqueous solutions or dispersions and sterile powders for the extemporaneous preparation thereof. Typical carriers therefor include biocompatible aqueous buffers, ethanol, glycerol, propylene glycol, polyethylene glycol and mixtures thereof. The pharmaceutical compositions of the invention may be suitably formulated, using formulating methods well known to those skilled in the art, for oral, intranasal, subcutaneous, intramusvular, intradermal, intravenous, intraarterial or parenteral administration or for catheterization.

The pharmaceutically acceptable carrier may also be a vector, preferably a retroviral vector and more preferably an adenovirus-assisted vector.

Gene delivery systems to heart and blood vessels by adenoviral vectors are known. These vectors do not integrate in the genome of the host cell. As these vectors rely on the division of their target cells, high titer retroviral stocks are needed for efficient gene delivery, as is described in U.S. Pat. No. 6,174,871. Preferred vectors for the delivery of genes to terminally differentiated cells of the heart and blood vessels are the Adenovirus-Assisted Virus (hereinafter AAV) vectors. In contrast to other gene delivery systems, these AAV vectors do not rely on the division of the target cells. In addition, AAV is not associated with any known mammalian pathology. AAV vectors integrate in the chromosome of the host cell. In addition they do not express the viral proteins that are expressed by classic adenoviral vectors. These foreign viral proteins can cause imflammation. As an example, the production and administration of an AAV vector is illustrated in U.S. Pat. No. 6,162,796. A review on the gene delivery in the cardiovascular system is given by Sinnaeve et al. in Cardiovasc Res. (1999) 44(3):498-506.

The pharmaceutical compositions of the invention may further comprise a therapeutically effective amount of at least one known thrombolytic agent, preferably in respective proportions such as to provide a synergistic effect in the said prevention or treatment. Thrombolytic agents which may namely be considered for this purpose include fibrin-specific agents such as wild-type tissue-type plasminogen activator and mutants and variants thereof, single-chain urokinase-type plasminogen activator and staphilokinase, and non-fibrin-specific agents such as two-chain urokinase-type plasminogen activator, streptokinase and anisoylated plasminogen streptokinase activator complex (anistreplase). All of them are well documented by R. Lijnen et al. in Cardiovascular Thrombosis (1998) pp.301-315, 2^{nd} ed., Linpicott-Raven Publishers (Philadelphia).

The method of prevention or treatment according to the invention may, in addition to administering a therapeutically effective amount of an inhibitor of a Gas6 function or of a Gas6 receptor, further comprise administering to the mammal a therapeutically effective amount of at least one known thrombolytic agent such as above described. The latter administration may be either simultaneous, separate or sequential with regard to administration of the Gas6 inhibiting component.

Alternatively, the Gas6 signalling cascade can be downregulated by a protease in the bloodstream that cleaves the extracellular domain (hereinafter ECD) of the Axl receptor. This cleavage is an in vivo phenomenon that modulates the Gas6 function at two levels according to O'Bryan et al. in J. Biol. Chem. (1995) 270, 2:551-557. The released ECD will bind to Gas6 and prevent signalling of Gas6. The membrane-bound intracellular domain of Axl is still functional as a kinase but is quickly degraded. The cleavage site in the Axl sequence has been mapped to a peptide of 14 amino acids (VKEPSTPAFSWPWW) which is amino-terminal to the transmembrane region. This sequence does not occur in any of the other receptors that can be regulated by proteolytic processing such as MET (hepatocyte -growth factor) and CSF1-R (colony stimulating factor 1 receptor). The Axl cleaving protease is further preferably characterized by activation by phorbol esters via Protein Kinase C. Consequently, the gas6 function can also be inhibited by administration of this Axl-ECD protease. A high dose will strip the cell of its Axl-ECD, therefore part of the Gas6 protein will be scavenged and the Axl receptor will be degraded.

Alternatively, Gas6 function can also be inhibited by inactivating the translation of Gas6, or a gas6 receptor such as Axl, Rse or mer, via antisense or ribozyme technology well known in the art. Antisense molecules may be used to modulate Gas6 or its receptor activity or to achieve regulation of the gene function. The disclosed polynucleotides encoding for Gas6 or its receptor antisense strands, or a vector containing these sequences may for instance be administered to a mammal in order to prevent or treat a disorder associated with platelet aggregation. A composition comprising therapeutically effective amounts of antisense strands to a polynucleotide (RNA) encoding Gas6 or its receptor may be mixed with any pharmaceutically acceptable carrier. The invention thus provides an antisense nucleic acid molecule that is complementary to at least a portion of the mRNA encoding a Gas6 or its receptor protein. Antisense nucleic acid molecules can be RNA or single-stranded DNA, and can be complementary to the entire mRNA molecule encoding Gas6 or its receptor or to only a portion thereof. These antisense molecules can be used to reduce levels of Gas6 or its receptor, for instance by introducing into cells an RNA or single-stranded DNA molecule that is complementary to at least a portion of the mRNA of Gas6 or its receptor (i.e. by introducing an antisense molecule). For a general discussion of antisense molecules and their use, reference is made to Rossi in Br. Med. Bull. (1995) 51:217-25. Classical antisense vectors producing antisense in the cytoplasma that will bind to the polyA mRNA transported from the nucleus into the cytoplasm may be used. A ratio of about 1000-100/1 antisense/sense RNA is usually required for an efficient inhibition. In addition the high amount of cytoplasmic RNA can provoke the response of interferons. Therefore, U.S. Pat. No. 5,908,779 discloses antisense vectors, which may also be used, wherein a modification prevents polyadenylation and the subsequent transport of the antisense RNA to the cytoplasm. This allows the antisense to function in the nucleus with a highly increased efficiency of inhibition of 5/1 ratio of antisense/sense RNA.

The invention further makes use of a special class of antisense RNA molecules, known as ribozymes, having recognition sequences complementary to specific regions of the mRNA encoding Gas6 or its receptor. Ribozymes not only complex with target sequences via complementary antisense sequences but also catalyze the hydrolysis, or cleavage, of the template mRNA molecule.

Expression of a ribozyme in a cell can inhibit gene expression. More particularly, a ribozyme having a recognition sequence complementary to a region of a mRNA encoding Gas6 or its receptor can be used to decrease expression of Gas6 or its receptor. A vector may be used for introduction of the ribozyme into a cell. In general the complementary sequence to a gene in the ribozyme construct can be much shorter than in antisense RNA molecules. In addition, even small mismatches or a limited number of mutated residues in the target RNA with respect to the complementary seqeunce in the ribozyme will prevent ribozyme assisted degradation. This makes the ribozyme much more specific over antisense RNA when highly related sequences to the target gene exist.

The present invention will be demonstrated in more detail in the following examples, which are however not intended to limit the scope of the invention.

### EXAMPLE 1

Mice Deficient in Gas6 (Gas6^{-/-} Mice) are Resistant to Stasis-Induced Thrombosis

Animal experiments were conducted according to the guiding principles of the American Physiological Society and the International Committee on Thrombosis and Haemostasis as disclosed by A. Giles in Thromb. Haemost. (1987) 58:1078-1084.

Thrombus formation by stasis was induced as described by Vogel et al. in Thromb. Res. (1989) 54:399-410. Briefly, wild type mice (Gas6^{+/+} mice) or mice in which Gas6 expression was abolished by homologous recombination (Gas6^{-/-} mice), of either sex, with a genetic background of 50% Swiss/50% 129, weighing 20 to 30 g, were anesthetized by intra-peritoneal injection of 60 mg/kg sodium pentobarbital. The abdomen of the animal was opened surgically and, after careful dissection, the vena cava was exposed and dissected free from surrounding tissue. Two loose sutures were prepared 0.7 cm apart on the inferior vena cava and all collateral veins were ligated. Stasis was established by tightening the two sutures, first the proximal and then the distal. The abdominal cavity was closed provisionally and stasis was maintained for 20 minutes. The cavity was then reopened, the ligated segment was opened longitudinally and the thrombus formed was removed, rinsed, blotted on filter paper, dried overnight at 60° C. and weighed.

The data are represented as mean±SEM of n determinations. The significance of differences was determined by unpaired t-test. In Gas6^{+/+} mice, thrombus weight was 2.46±0.24 mg (n=10). In contrast, thrombus weight measured in Gas6^{-/-} mice was 0.38±0.08 mg (n=10) (84.6% inhibition in Gas6^{-/-} mice, p<0.001). These data indicate that the lack of Gas6 expression induced resistance to stasis-induced thrombosis.

### EXAMPLE 2

Mice Deficient in Gas6 (Gas6^{-/-} Mice) are Resistant to Thrombosis in the Carotid Artery Photochemically Induced by Rose-Bengal

Mice were anesthetized by intraperitoneal injection of 60 mg/kg sodium pentobarbital and then fixed on heated operating table. Atropine sulphate was injected in all animals subcutaneously (0.5 mg/kg), and endotracheal intubation was carried out. A 2F venous catheter was inserted into the right jugular vein for injection of rose bengal. The left carotid artery was exposed and mounted on an appropriate transilluminator. Thrombus formation was induced by a photochemical reaction according to the method of Umemura et al. in Thromb. Haemost. (1996) 76:799-806.

Briefly, the exposed artery was irradiated with green light (wavelength 540 nm) of a Xenon lamp (L4887, Hamamatsu Photonics, Hamamatsu, Japan) equipped with a heat-absorbing filter and a green filter. Irradiation was directed via a 3 mm diameter optic fiber attached to a manipulator. Rose bengal was administered via an intravenous (slow) bolus injection in a total volume of 200 □I. Irradiation was started just after injection and was maintained for 4 minutes according to Kawasaki et al. in Throm. Haemost. (1999) 81:306-11.

In Gas6^{+/+} mice, thrombus mass was 380±91×10³ light units (mean±SEM, n=5). In contrast, thrombus mass measured in Gas6^{-/-} mice was 160±35×10³ light units (mean±SEM, n=5), (p<0.05). These data indicate that the lack of Gas6 expression induced resistance to arterial thrombosis photochemically induced by rose bengal.

### EXAMPLE 3

Mice Deficient in Gas6 (Gas6^{-/-} Mice) are Protected Against Collagen/Epinephrin-Induced Thromboembolism

A mixture of collagen (0.5 mg/kg, equine collagen, Kollagenreagent Horm, available from Hormon Chemie, Munich, Germany) and epinephrine (60 □g/kg) was injected into the jugular vein of mice anesthetized by intraperitoneal injection of 60 mg/kg sodium pentobarbital according to Di Minnio et al. in J. Pharmacol Exp. Ther. (1983) 225:57-60.

The mortality within 15 minutes induced by infusion of a collagen/epinephrin mixture was 80% in Gas6^{+/+} mice (n=10) versus 20% in GAS6^{-/-} mice (n=10) (p<0.03). These data indicate that the lack of Gas6 expression induced resistance to collagen/epinephrin thromboembolism.

### EXAMPLE 4

Platelet Aggregation is Defective in Gas6 Deficient Mice (Gas6^{-/-} Mice)

From mice anesthetized by intraperitoneal injection of 60 mg/kg sodium pentobarbital, whole blood was drawn from the inferior vena cava into 0.1 M citrate (1 volume anticoagulant/9 volumes of blood). Blood was centrifuged at 100 g for 10 minutes, allowing separation of platelet-rich plasma (PRP). Platelet-poor plasma (PPP) was obtained by centrifugation of the remaining blood at 2,000 g for 10 minutes. PRP and PPP were pooled from four Gas6^{-/-} or Gas6^{+/+} mice. Platelet aggregation was measured turbidimetrically in an optical Chronolog aggregometer (model 490, Coulter Electronics Ltd), using 280 μl PRP, adjusted to a concentration of 250,000 platelets/μl with PPP as a diluent. PPP also served as 100% reference for aggregation. Aggregation in response to collagen (equine collagen from Hormon Chemie), ADP or the thromboxane A2 mimetic U46619 was studied.

Aggregation in response to thrombin was performed with washed platelets. Briefly, blood was drawn from the inferior vena cava into acid-citrate-dextrose solution (ACD) (1 volume ACD/6 volume blood) and PRP was pooled from four Gas6^{-/-} or Gas6^{+/+} mice. Apyrase was added to PRP at a final concentration of 1 u/ml. Platelets were then washed by adding 2 volumes ACD and pelleted by centrifugation at 2,000 g for 10 minutes. Platelet pellet was resuspended in Tyrode's buffer containing 1% BSA and final platelet suspension was adjusted to 200,000 platelets/μl and kept at 37° C. Platelet aggregation was measured with an optical Chronolog aggregometer as described above.

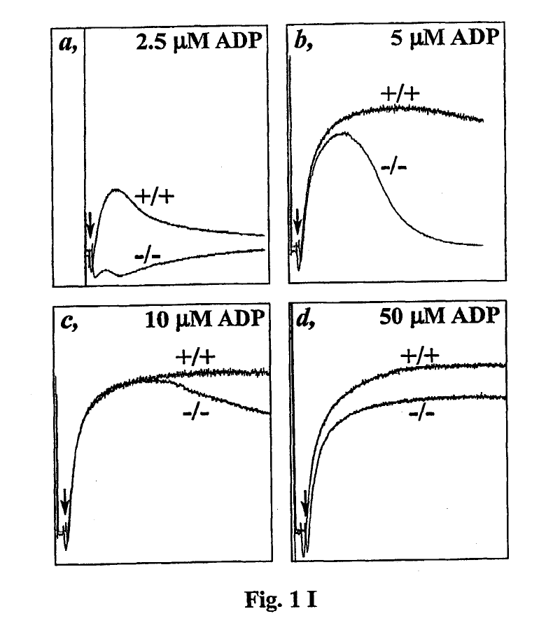

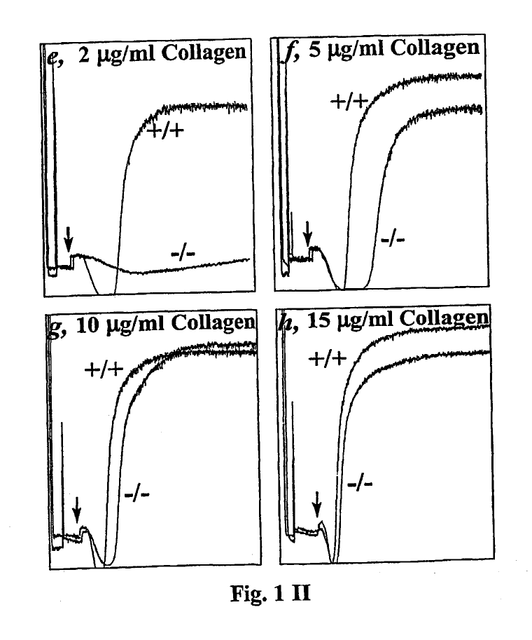

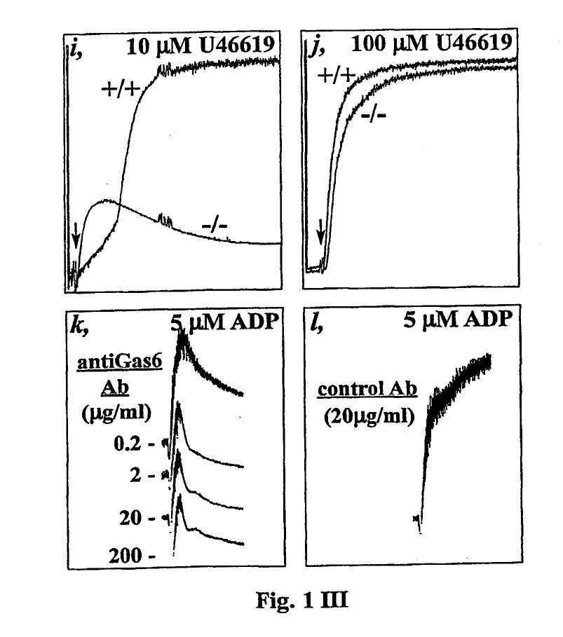

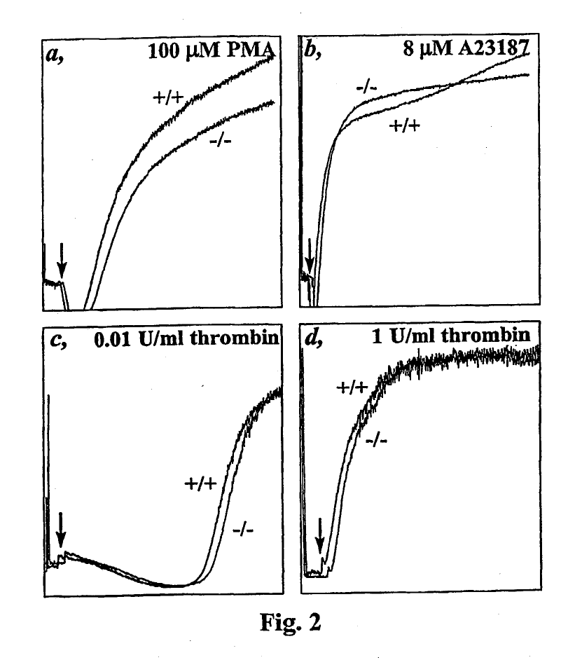

As shown in FIGS. 1 and 2, platelet aggregation studies revealed significant functional defects in Gas6^{-/-} mice. Platelets from Gas6^{+/+} mice dose-dependently aggregated in response to ADP (FIG. 1a-d), collagen (FIG. 1e-h) or the thromboxane A₂ analogue U46619 (FIG. 1i,j). Maximal aggregation was achieved at similar concentrations as used previously by Offermans et al. in Nature (1997) 389:183-186. In contrast, platelets from Gas6^{-/-} mice failed to irreversibly aggregate in response to low concentrations of ADP (<10 μM), collagen (2 μg/ml) and U46619 (10 μM). At low agonist concentration, Gas6^{-/-} platelets only displayed shape change as revealed by an immediate decrease in light transmission after stimulation. However, higher concentrations of ADP (50 μM in FIG. 1d), collagen (5-15 μg/ml in FIG. 1f-h) or U46619 (100 μM in FIG. 1j) induced irreversible aggregation of Gas6^{-/-} platelets. Both Gas6+/+and Gas6^{-/-} platelets aggregated normally in response to phorbol-12-myristyl-13-acetate (PMA) or the Ca⁺⁺ ionophore A23187 (FIG. 2a,b). Thrombin stimulated platelet aggregation comparably in both genotypes at all doses tested (FIG. 2c,d). In both FIGS. 1 and 2, squares represent 2 minutes (on X-axis) and 10% change in light transmission (on Y-axis). The arrow in each panel indicates the application of the platelet agonists (in FIG. 1), PMA or thrombin (in FIG. 2).

### EXAMPLE 5

Platelet Secretion is Decreased in Gas6^{-/-} Mice

Platelet aggregation and, ATP secretion were measured in an optical Chronolog Lumi-aggregometer (Coulter Electronics Ltd), using 280 μl PRP, adjusted to a concentration of 250,000 platelets/μl with PPP as a diluent. Platelet aggregation was measured as described in example 4. Platelet ATP release was monitored by adding firely luciferase and luciferin to all samples and comparing luminescence created by platelet ATP release to that generated by addition of an ATP standard (Chrono-Lume, Kordia). Aggregation and ATP release in response to collagen (equine collagen from Hormon Chemie), ADP, or the thromboxane A2 mimetic U46619 were studied. Platelet aggregation and ATP secretion in response to thrombin were performed with washed platelets in an optical Chronolog Lumi-aggregometer as described above.

Secretion of ADP and ATP from dense granules is essential for the formation of stable macroaggregates after initial formation of small, unstable platelet aggregates as described by A. J. Marcus, Disorders of Hemostasis (1997) 79-137 (eds. Ratnoff & Forbes). Secretion of dense granule stores (evaluated by measuring release of ATP) was significantly impaired in Gas6^{-/-} platelets. Compared to Gas6^{+/+} platelets, release of ATP from Gas6^{-/-} platelets was significantly decreased in response to ADP, collagen or U46619, when these agonists were used at low concentrations (only causing platelet shape changes or reversible platelet aggregation) as shown in following Table 1. ATP release from Gas6^{-/-} platelets was also reduced in response to high concentration of ADP (50 μM) or thrombin (1 U/ml). However, release of ATP was normal or only slightly reduced when Gas6^{-/-} platelets were stimulated with high concentrations of collagen (10 μg/ml) or U46619 (100 μM) (see Table 1), which cause irreversible platelet aggregation. PMA and the Ca⁺⁺ ionophore A23187 induced a normal secretion response in both genotypes (see Table 1). Thus, a close correlation was found between the defects in the aggregation (see also example 4) and ATP secretion response of Gas6^{-/-} platelets to various agonists.

TABLE 1

| | concentra- | | |
|---|---|---|---|
| Agonist | tion | Gas6^{+/+} | Gas6^{-/-} |
| ADP | 20 μM | 0.8 ± 0.1 | 0.2 ± 0.1* |
| | 50 μM | 2.6 ± 0.8 | 1.3 ± 0.3* |
| Collagen | 1 μg/ml | 1.8 ± 0.3 | ND |
| | 10 μg/ml | 10 ± 1.0 | 11 ± 0.8 |
| U46619 | 10 μM | 6.5 ± 1.5 | ND |
| | 100 μM | 8.2 ± 2.0 | 7.6 ± 2.3 |
| Thrombin | 1 U/ml | 17 ± 1.6 | 11 ± 1.2* |
| PMA | 100 μM | 2.3 ± 0.4 | 1.9 ± 0.6 |
| A23187 | 8 μM | 9.2 ± 3.1 | 7.3 ± 3.0 |

In Table 1, the data represent mean±SEM of three experiments using platelet-rich plasma (for ADP, collagen, U46619, PMA or A23187 stimulation) or washed platelets (for thrombin stimulation). Each experiment was performed with a pool of 4 to 6 Gas6+/+or Gas6^{-/-} mice. ND means not detectable.

### EXAMPLE 6

Anti-Gas6 Antibodies Inhibit Platelet Aggregation

Blood from human volunteers was collected from the antecubital vein (9 volumes) and anticoagulated with 3.8% citrate (1 volume). PRP was obtained by centrifugation of citrated whole blood at 120 g for 15 minutes. Washed platelets were prepared as mentioned in example 4. The effect of Gas6-specific antibodies was studied on platelet aggregation in vitro. Antibodies (available from Santa Cruz Biotechnology, Santa Cruz, Calif.) directed against the carboxyterminal part of Gas6—responsible for binding of Gas6 to its receptors according to Mark et al. in J.Biol.Chem. (1996) 271:9785-9—were used. Platelet aggregation was measured turbidimetrically in an optical Chronolog aggregometer (model 490, Coulter Electronics Ltd), using 280 μl PRP, adjusted to a concentration of 250,000 platelets/μl with PPP as a diluent. PPP also served as 100% reference for aggregation. Platelets were pre-incubated with Gas6 neutralizing antibodies (at concentrations of 0.2, 2, 20 and 200 μg/ml respectively) or isotype-matched control antibodies (at a concentration of 20 μg/ml) for 15 seconds at 37° C. before induction of aggregation with ADP (5 μM). Gas6 neutralizing antibodies dose-dependently blocked aggregation of washed human platelets in response to ADP (5 μM) as shown in FIG. 1k-l. None of the antibodies had any effect on platelets in the absence of ADP.

### EXAMPLE 7

Anti-Gasμ6 Antibodies Protect Mice Against Collagen/Epinephrin-Induced Thromboembolism

Mice where injected through the tail vein with either 100 μg goat polyclonal antibodies directed against the carboxyterminal part of human Gas6 or irrelevant isotype-matched antibodies 30 minutes before the thromboembolism challenge. Thromboembolism was then induced by injecting a mixture of collagen (0.5 mg/kg, equine collagen from Hormon Chemie) and epinephrine (60 μg/kg) into the jugular vein of mice anesthetized by intraperitoneal injection of 60 mg/kg sodium pentobarbital.

The mortality within 15 minutes induced by infusion of a collagen/epinephrine mixture was 80% in wild-type mice pre-injected with irrelevant isotype-matched antibodies (n=16) versus 25% in wild-type mice pre-injected with anti-Gas6 antibodies (n=12) (p<0.03). Thus, Gas6-neutralizing antibodies protected wild-type mice against fatal collagen/epinephrine-induced thromboembolism to the same degree as genetic loss of Gas6. Gas6 antibody-treated mice did not show any signs of bleeding. These results indicate that inhibition of Gas6 effectively blocks thrombosis.

### EXAMPLE 8

Gas6 Deficient Mice (Gas6^{-/-} Mice) Have no Spontaneous Bleeding and Normal Bleeding Time

No spontaneous bleeding tendency has been observed in Gas6^{-/-} mice. The morphology of Gas6^{-/-} platelets on blood smear was normal. Bleeding time (performed by tail transsection according to Dejana et al. in Thromb. Haemost. (1982) 48:108-111) was comparable in Gas6^{+/+} and Gas6^{-/-} mice (166±48 μl, n=10 and 172±68 μl, n=10, respectively, p>0.05). Thus, inhibitors of Gas6 function and of Gas6 receptors, in particular tyrosine kinase receptors such as the Axl, the Rse or the c-Mer receptor, appear to represent a new class of anti-thrombotic drugs with a favorable antithrombotic/bleeding ratio.

1. Use of a composition comprising:
(a) an inhibitor of a Gas6 function or of a Gas6 receptor, or a ribozyme or an antisense RNA directed against Gas 6 or a Gas 6 receptor function, or a protease able to cleave the extracellular domain of the Axl receptor, and
(b) a thrombolytic agent for the manufacture of a medicine for the prevention or treatment of a thromboembolic disease or a thrombotic pathologic condition in a mammal, in respective proportions such as to provide a synergistic effect in the said prevention or treatment.

2. A pharmaceutical composition comprising an inhibitor of a Gas6 function or of a Gas6 receptor, or a ribozyme or an antisense RNA directed against Gas 6 or a Gas 6 receptor function, or a protease able to cleave the extracellular domain of the Axl receptor, as an active ingredient in admixture with at least a pharmaceutically acceptable carrier.

3. A pharmaceutical composition according to claim 2, wherein the said Gas6 receptor is a tyrosine kinase receptor.

4. A pharmaceutical composition according to claim 2 or claim 3, wherein the said Gas6 receptor is selected from the Axl receptor, the Rse receptor, the c-Mer receptor and fragments thereof.

5. A pharmaceutical composition according to any of claims 2 to 4, wherein the said inhibitor is a Gas6 function neutralizing antibody.

6. A pharmaceutical composition according to claim 2, wherein the said protease is able to cleave the extracellular domain of the Axl receptor within the sequence VKEPSTPAFSWPWW.

7. A pharmaceutical composition according to claim 2 or claim 6, wherein the said protease is in vivo activated by phorbol esters.

8. A pharmaceutical composition according to any of claims 2 to 7 for the prevention or treatment of a cardiovascular disease.

9. A pharmaceutical composition according to claim 8, wherein the cardiovascular disease is other than resulting from an endothelial dysfunction.

10. A pharmaceutical composition according to claim 8 or claim 9, wherein the cardiovascular disease is caused by platelet aggregation.

11. A pharmaceutical composition according to any of claims 8 to 10, wherein the cardiovascular disease is a thromboembolic disease or a thrombotic pathologic condition in a mammal.

12. A pharmaceutical composition according to claim 11, wherein the said thromboembolic disease or thrombotic pathologic condition is selected from an ischemic disease, ischemic stroke, ischemic cerebral infarction, acute myocardial infarction, chronic ischemic heart disease, an ischemic disease of an organ other than myocardium or a region of the brain, venous thromboembolism, arterial or venous thrombosis, pulmonary embolism, restenosis following coronary artery bypass surgery or following percutaneous transluminal angioplasty of coronary artery.

13. A pharmaceutical composition according to any of claims 2 to 12, wherein the said pharmaceutically acceptable carrier is a vector, preferably a retroviral vector and more preferably an adenovirus-assisted vector.

14. A pharmaceutical composition according to any of claims 2 to 13, further comprising a thrombolytic agent in respective proportions such as to provide a synergistic effect in the prevention or treatment of a thromboembolic disease or a thrombotic pathologic condition, as a combined preparation for simultaneous, separate or sequential use in therapy.

15. Use of inhibition of a Gas6 function or of a Gas6 receptor for the prevention or treatment of a cardiovascular disease other than resulting from an endothelial dysfunction.

16. Use according to claim 15, wherein the cardiovascular disease is caused by platelet aggregation.

17. Use according to claim 15 or claim 16, wherein the cardiovascular disease is a thromboembolic disease or a thrombotic pathologic condition in a mammal.

18. Use according to any of claims 15 to 17, wherein inhibition is effected by means of an inhibitor or antagonist of a Gas6 function or a Gas6 receptor, or by means of a ribozyme or an antisense RNA directed against Gas 6 or a Gas 6 receptor function.

19. Use according to any of claims 15 to 18, wherein the said Gas6 receptor is a tyrosine kinase receptor.

20. Use according to any of claims 15 to 19, wherein the said Gas6 receptor is selected from the Axl receptor, the Rse receptor, the c-Mer receptor and fragments thereof.

21. Use according to any of claims 15 to 20, wherein the said inhibitor is a Gas6 function neutralizing antibody.

22. Use according to any of claims 17 to 21, wherein the said thromboembolic disease or thrombotic pathologic condition is selected from an ischemic disease, ischemic stroke, ischemic cerebral infarction, acute myocardial infarction, chronic ischemic heart disease, an ischemic disease of an organ other than myocardium or a region of the brain, venous thromboembolism, arterial or venous thrombosis, pulmonary embolism, restenosis following coronary artery bypass surgery or following percutaneous transluminal angioplasty of coronary artery.

23. Use according to any of claims 15 to 18, wherein inhibition of the Gas6 function is effected by means of a protease able to cleave the extracellular domain of the Axl receptor.

24. Use according to claim 23, wherein the said protease is able to cleave the extracellular domain of the Axl receptor within the sequence VKEPSTPAFSWPWW.

25. Use according to claim 23 or claim 24, wherein the said protease is in vivo activated by phorbol esters.

26. Use of an inhibitor of a Gas6 function or of a Gas6 receptor during extracorporeal blood circulation and hemodialysis.

27. Use according to claim 26 in order to identify, via protein or mRNA or DNA characterization, individuals having a predisposition to acquire a thromboembolic disease or a thrombotic pathologic condition.

28. Use of an inhibitor of a Gas6 function or of a Gas6 receptor as a diagnostic agent.

29. Use according to claim 28, wherein the said inhibitor is a Gas6 function neutralizing antibody.

30. A method of prevention or treatment of a cardiovascular disease resulting from a dysfunction other than an endothelial dysfunction in a mammal, comprising administering to a mammal in need of such prevention or treatment a therapeutically effective amount of an inhibitor of a Gas6 function or of a Gas6 receptor, or a ribozyme or an antisense RNA directed against Gas 6 or a Gas 6 receptor function, or a protease able to cleave the extracellular domain of the Axl receptor.

31. A method of prevention or treatment according to claim 30, wherein the cardiovascular disease is caused by platelet aggregation.

32. A method of prevention or treatment according to claim 30 or claim 31, wherein the cardiovascular disease is a thromboembolic disease or a thrombotic pathologic condition.

33. A method of prevention or treatment according to claim 32, wherein the said thromboembolic disease or thrombotic pathologic condition is selected from an ischemic disease, ischemic stroke, ischemic cerebral infarction, acute myocardial infarction, chronic ischemic heart disease, an ischemic disease of an organ other than myocardium or a region of the brain, venous thromboembolism, arterial or venous thrombosis, pulmonary embolism, restenosis following coronary artery bypass surgery or following percutaneous transluminal angioplasty of coronary artery.

34. A method of prevention or treatment according top any of claims 30 to 33, wherein the said Gas6 receptor is a tyrosine kinase receptor.

35. A method of prevention or treatment according top any of claims 30 to 34, wherein the said Gas6 receptor is selected from the Axl receptor, the Rse receptor, the c-Mer receptor and fragments thereof.


SEQUENCE LISTING (1 sequence)

SEQ ID NO: 1 - protein, 14 aa, Homo sapiens
  MISC_FEATURE (1)..(14): fragment of human Axl receptor tyrosine kinase NP_068713(amino acids 438-451)
VKEPSTPAFSWPWW